(19) United States
(12) Patent Application Publication
(10) Pub. No.: US 2024/0059740 A1
(43) Pub. Date: Feb. 22, 2024

(54) CYCLIC PEPTIDES AND USES THEREOF

(71) Applicant: PREVECEUTICAL MEDICAL INC., Vancouver (CA)

(72) Inventors: Harendra PAREKH, Chapel Hill, Queensland (AU); Stephen Van DEVENTER, Vancouver (CA); Karnaker TUPALLY, St Lucia, Queensland (AU)

(21) Appl. No.: 18/268,951

(22) PCT Filed: Dec. 22, 2021
(86) PCT No.: PCT/AU2021/051545

§ 371 Date: Jun. 21, 2023

(30) Foreign Application Priority Data
Dec. 22, 2020 (AU) 2020904798

(51) Int. Cl. C07K 7/64; A61P 35/04

(52) U.S. Cl. CPC C07K 7/64; A61P 35/04

(57) ABSTRACT

Cyclic peptides and their use in methods of treating or preventing cancer, such as brain cancer. Pharmaceutical compositions including the cyclic peptides are also described.

### FIELD OF THE INVENTION

The present invention relates to cyclic peptides and their use in methods of treating or preventing cancer such as brain cancer. Pharmaceutical compositions comprising the cyclic peptides are also described.

### BACKGROUND OF THE INVENTION

Gliomas represent 40 to 45% of all intracranial tumours and are characterized by their potent ability to infiltrate into surrounding normal brain tissue, making them a challenge to treat (Kleihues, P., et al., 1995). Invasion is a process that involves adhesion to the extracellular matrix (ECM), degradation of the ECM components and basement membrane by matrix metalloproteinases (MMPs) and migration through the digested ECM (Goldbrunner, R et al., 1999). Glioblastoma multiforme (GBM) is the most common, and fatal form of brain cancer, affecting adults between 45 and 60 years of age. Current treatments for GBM include chemotherapy, radiotherapy and surgery. However, the average survival time for most patients is approximately one year after diagnosis (Dai, C et al., 2001). A major feature of GBM that contributes to poor prognosis is its high level of invasiveness. The breakdown of the surrounding tissues is essential to the progression of GBM, thus degradation of ECM constituents can be used as a hallmark of tumour malignancy in vitro (Alves, T. R., et al., 2011). Recent reports show that MMPs play pivotal roles in invasiveness of GBM by the following possible mechanisms: MMPs can degrade ECM and basement membrane, activate signal transduction, release ECM-bound growth factors, activate growth factors, increase tumour cell motility, and promote angiogenesis (McCawley and Matrisian, 2001; Conlon and Murray, 2019). Both expression of MMP-2 and MMP-9 is raised in GBM. Multiple studies have reported that the expression of higher level of MMPs in brain tumours is associated with increased tumour aggressiveness (Nakagawa, T., et al., 1996). MMPs are upregulated in nearly all human and animal tumours as well as in many tumour cell lines.

In glioma, it is the brain tissue itself that drives the disease, and it transforms it into invasive phenotype, which is the key driver of is spread and poor prognosis. There is a need to find means of reducing expression of and/or activity of MMPs in the extracellular matrix to reduce the invasive properties of glioma and reduce the need to resect brain tissue.

A peptide, chlorotoxin, derived from the Deathstalker scorpion has been extensively studied and shows promise in delineating glioma tissue and also preventing growth, invasion and metastasis of gliomas through inhibition of extracellular matrix metalloproteases (MMP). However, chlorotoxin is a complex cyclic peptide comprising 36 amino acids and four disulfide linkages.

There is a need for simpler, yet effective, modulators of MMPs to treat invasive cancers such as brain cancer.

### SUMMARY OF THE INVENTION

The present invention is predicated at least in part on the discovery that simple cyclic peptides modelled on cytotoxic peptides isolated from Cuban Blue Scorpion venom have MMP inhibitory activity similar to chlorotoxin.

In one aspect, the present invention provides a compound represented by the formula (I):

R₁-Xaa₁-Pro-c(Xaa₂-Xaa₃-Xaa₄-Xaa₅-Xaa₆-Xaa₇-Xaa₈-Xaa₉)-Xaa₁₀-R₂ (I)

wherein
R₁ is acyl or hydrogen;
R₂ is OH or NH₂;
Xaa₁ is a hydrophobic amino acid residue,
Xaa₂ is selected from SSA, Asp, Glu and Cys;
Xaa₃ is an amino acid residue having a small side chain;
Xaa₄ is a hydrophobic amino acid residue;
Xaa₅ is a polar uncharged amino acid residue;
Xaa₆ is a negatively charged amino acid residue;
Xaa₇ and Xaa₈ are each independently absent or an amino acid with a small side chain;
Xaa₉ is selected from SSA, Asp, Glu and Cys; and
Xaa₁₀ is a positively charged amino acid residue;
   wherein one of the following applies:
i) Xaa₂ is SSA and Xaa₉ is Asp or Glu;
ii) Xaa₂ is Asp or Glu and Xaa₉ is SSA; or
iii) Xaa₂ and Xaa₉ are both Cys;
   wherein Xaa₂ and Xaa₉ are linked to form a cyclic peptide; and wherein SSA is an amino acid residue of formula (II):

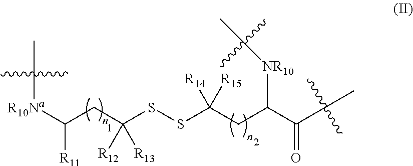

wherein
each R₁₀ is independently selected from hydrogen, substituted or unsubstituted C₁₋₆ alkyl;
R₁₁ is selected from hydrogen and methyl;
R₁₂, R₁₃, R₁₄ and R₁₅ are each independently selected from the group consisting of hydrogen and C₁₋₄ alkyl, or R₁₂ and R₁₃ taken together with the carbon atom to which they are attached and/or R₁₄ and R₁₅ taken together with the carbon atom to which they are attached for a cyclic structure selected from C₃₋₅ cycloalkyl and C₃₋₅ oxygen-containing, nitrogen containing or sulfur-containing heterocycle, each of which may be substituted or unsubstituted; and
n₁ and n₂ are each independently 0, 1, 2, 3 or 4;
with the proviso that at least one of R₁₂, R₁₃, R₁₄ and R₁₆ is not hydrogen;
wherein SSA is linked to Xaa₂ or Xaa₉ via N^{a};
or a pharmaceutically acceptable salt thereof.

In another aspect of the present invention there is provided a method of treating or preventing cancer comprising administering a compound of formula (I) or a pharmaceutically acceptable salt thereof as described herein. In particular embodiments, the cancer is a central nervous system cancer.

In another aspect of the invention there is provided a method of inhibiting urokinase plasminogen activator and/or a matrix metalloproteinase comprising contacting the urokinase plasminogen activator and/or matrix metalloprotease with a compound of formula (I) or a pharmaceutically acceptable salt thereof.

In a further aspect, there is provided a method of inhibiting tumour cell invasion in a brain tumour comprising administering to the brain tumour a compound of formula (I) or a pharmaceutically acceptable salt thereof.

### BRIEF DESCRIPTION OF THE FIGURE

FIG. 1 provides fluorescence staining of biotinylated peptide SEQ ID NO.1 after 6 hours cell exposure (U251 cells and U87 cells) or 5 min cell exposure (081024 oncospheres). Upper right hand panel: U251, middle right hand panel: U87, lower right hand panel: 081024. no peptide negative control (left side of each panel). Cells were fixed, permeabilized and the biotinylated peptides revealed by binding of Cy3 Avidin (shown in red). Nuclei appear blue due to DAPI staining. Images were captured using a 100× objective and an Olympus FV3000 confocal microscope.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 20%, 15% or 10% to a reference quantity, level, value, dimension, size, or amount.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “amino acid” refers to an α-amino acid or a β-amino acid and may be a L- or D-isomer. The amino acid may have a naturally occurring side chain (see Table 1) or a non-proteinogenic side chain (see Table 2). The amino acid may also be further substituted in the α-position or the β-position with a group selected from —C₁₋₆alkyl, —(CH₂)ₙCORₐ, —(CH₂)ₙR_{b} and —PO₃H, where Rₐ is —OH, —NH₂, —NHC₁₋₃alkyl, —OC₁₋₃alkyl or —C₁₋₃alkyl and R_{b} is —OH, —SH, —SC₁₋₃alkyl, —OC₁₋₃alkyl, —NH₂, —NHC₁₋₃alkyl or —NHC(C═NH)NH₂ and where each alkyl group may be substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl.

Amino acid structure and single and three letter abbreviations used throughout the specification are defined in Table 1, which lists the twenty naturally occurring amino acids which occur in proteins as L-isomers.

TABLE 1

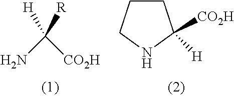

| | Three-letter | One-letter | |
|---|---|---|---|
| Amino Acid | Abbreviation | symbol | Structure of side chain (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indoly1) |
| Tyrosine | Tyr | Y | CH₂(4-hydroxyphenyl) |
| Valine | Val | V | —CH(CH₃)₂ |

The term “non-proteinogenic amino acid” as used herein, refers to amino acids having a side chain that does not occur in the naturally occurring L-α-amino acids recited in Table 1. Examples of non-proteinogenic amino acids and derivatives include, but are not limited to, norleucine, 4-aminobutyric acid, 4-amino-3-hydroxy-5-phenylpentanoic acid, 6-aminohexanoic acid, t-butylglycine (Tbg), norvaline, phenylglycine, ornithine (Orn), citrulline (Cit), sarcosine (Sar), 4-amino-3-hydroxy-6-methylheptanoic acid, 2-thienyl alanine and/or D-isomers of natural amino acids. A list of unnatural amino acids that may be useful herein is shown in Table 2

TABLE 2

| Amino Acid | Structure |
|---|---|
| sarcosine | 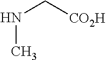 |
| citrulline | 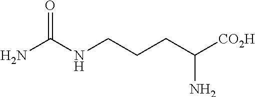 [SMILES: NC(=O)NCCCC(N)C(=O)O] |
| ornithine | 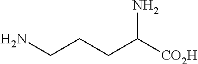 |
| tert-butyl glycine | 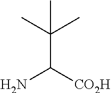 |
| 4(γ)-amino- butyric acid | 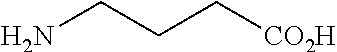 |
| norvaline | 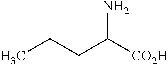 |
| phenylglycine | 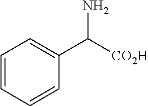 [SMILES: NC(C(=O)O)c1ccccc1] |
| diaminobutyric acid | 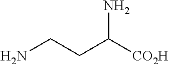 |
| homoarginine | 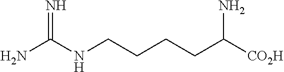 [SMILES: N=C(N)NCCCCC(N)C(=O)O] |
| Cyclopentyl alanine | 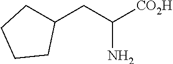 [SMILES: NC(CC1CCCC1)C(=O)O] |
| cyclohexyl- alanine | 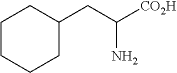 [SMILES: NC(CC1CCCCC1)C(=O)O] |
| norleucine | 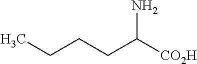 |

The non-proteinogenic amino acids in Table 2 may be in the L or D configuration and may be N-methylated on the α-amino group.

The term “alkyl” as used herein refers to straight chain or branched hydrocarbon groups, for example, alkyl groups may have 1 to 20 carbon atoms, such as 1 to 10 carbon atoms. Suitable alkyl groups include, but are not limited to methyl, ethyl, propyl, isopropyl, butyl, isobutyl, pentyl and hexyl. The term alkyl may be prefixed by a specified number of carbon atoms to indicate the number of carbon atoms or a range of numbers of carbon atoms that may be present in the alkyl group. For example, C₁₋₃alkyl refers to methyl, ethyl, propyl and isopropyl.

The term “cycloalkyl” used herein refers to a cyclic alkyl group having a specified number of carbon atoms. For example, C₃₋₅cycloalkyl includes cyclopropyl and cyclobutyl, cyclopentyl.

The term “heterocyclyl” used herein refers to a cycloalkyl group in which one or more carbon atoms have been replaced with an oxygen, nitrogen or sulfur atom. The heterocyclyl group may have a specified number of atoms in the ring. For example, C₃₋₅heterocyclic groups include ethylene oxide, thiirane, aziridine, oxetane, azetidine, thietane, tetrahydrofuran, pyrrolidine and thiolane.

As used herein, the term “optionally substituted” or “optional substituent” includes substitution with a substituent selected from C₁₋₆alkyl, C₂₋₆alkenyl, C₃₋₆cycloalkyl, oxo (═O), —OH, —SH, C₁₋₆alkylO—, C₂₋₆alkenylO—, C₃₋₆cycloalkylO—, C₁₋₆alkylS—, C₂₋₆alkenylS—, C₃₋₆cycloalkylS—, —CO₂H, —CO₂C₁₋₆alkyl, —NH₂, —NH(C₁₋₆alkyl), —N(C₁₋₆alkyl)₂, —NH(phenyl), —N(phenyl)₂, —CN, —NO₂, -halogen, —CF₃, —OCF₃, —CF₃, —CHF₂, —OCHF₂, —SCHF₂, -phenyl, —Ophenyl, —C(O)phenyl, —C(O)C₁₋₆alkyl. Examples of suitable substituents include, but are not limited to, methyl, ethyl, propyl, isopropyl, butyl, sec-butyl, tent-butyl, vinyl, methoxy, ethoxy, propoxy, isopropoxy, butoxy, methylthio, ethylthio, propylthio, isopropylthio, butylthio, hydroxy, hydroxymethyl, hydroxyethyl, hydroxypropyl, hydroxybutyl, fluoro, chloro, bromo, iodo, cyano, nitro, —CO₂H, —CO₂CH₃, trifluoromethyl, trifluoromethoxy, trifluoromethylthio, difluoromethyl, difluoromethoxy, difluoromethylthio, morpholino, amino, methylamino, dimethylamino, phenyl, phenoxy, phenylcarbonyl, benzyl and acetyl.

The term “hydrophilic amino acid residue” as used herein refers to an amino acid residue in which the side chain is polar or charged. Examples include glycine, sarcosine (N-methylglycine), L-serine, L-threonine, L-cysteine, L-tyrosine, L-asparagine, L-glutamine, L-aspartic acid, L-glutamic acid, L-lysine, L-arginine, L-histidine, L-ornithine, D-serine, D-threonine, D-cysteine, D-tyrosine, D-asparagine, D-glutamine, D-aspartic acid, D-glutamic acid, D-lysine, D-arginine, D-histidine, D-ornithine, N-methyl-L-serine, N-methyl-L-threonine, N-methyl-L-cysteine, N-methyl-L-tyrosine, N-methyl-L-asparagine, N-methyl-L-glutamine, N-methyl-L-aspartic acid, N-methyl-L-glutamic acid, N-methyl-L-lysine, N-methyl-L-arginine, N-methyl-L-histidine, N-methyl-L-ornithine, N-methyl-D-serine, N-methyl-D-threonine, N-methyl-D-cysteine, N-methyl-D-tyrosine, N-methyl-D-asparagine, N-methyl-D-glutamine, N-methyl-D-aspartic acid, N-methyl-D-glutamic acid, N-methyl-D-lysine, N-methyl-D-arginine, N-methyl-D-histidine and N-methyl-D-ornithine, L-diaminobutyric acid, D-diaminobutyric acid, N-methyl-L-diaminobutyric acid, N-methyl-D-diaminobutyric acid, L-citrulline, D-citrulline, N-methyl-L-citrulline, N-methyl-D-citrulline, L-homoarginine, D-homoarginine, N-methyl-L-homoarginine, N-methyl-D-homoarginine, L-O-methyltyrosine, D-O-methyltyrosine, N-methyl-L-O-methyltyrosine, N-methyl-D-O-methyltyrosine, L-homotyrosine, D-homotyrosine, N-methyl-L-homotyrosine, N-methyl-D-homotyrosine, L-O-methylhomotyrosine, D-O-methylhomotyrosine, N-methyl-L-O-methylhomotyrosine, N-methyl-D-O-methylhomotyrosine, L-4-carboxyphenylalanine, D-4-carboxyphenylalanine, L-N-methyl-4-carboxyphenylalanine and D-N-methyl-4-carboxyphenylalanine.

As used herein, the term “hydrophobic amino acid residue” refers to an amino acid residue in which the side chain is non-polar. Examples include, but are not limited to L-alanine, L-valine, L-leucine, L-isoleucine, L-proline, L-methionine, L-phenylalanine, L-tryptophan, L-γ-aminoisobutyric acid, D-alanine, D-valine, D-leucine, D-isoleucine, D-proline, D-methionine, D-phenylalanine, D-tryptophan, D-γ-aminoisobutyric acid, L-cyclohexylalanine, D-cyclohexylalanine, L-cyclopentylalanine, D-cyclopentylalanine, L-norleucine, D-norleucine, L-norvaline, D-norvaline, L-tert-butylglycine, D-tert-butylglycine, L-ethylglycine, D-ethylglycine, 6-aminohexanoic acid, 8-aminooctanoic acid, N-methyl-L-alanine, N-methyl-L-valine, N-methyl-L-leucine, N-methyl-L-isoleucine, N-methyl-L-proline, N-methyl-L-methionine, N-methyl-L-phenylalanine, N-methyl-L-tryptophan, N-methyl-L-γ-aminoisobutyric acid, N-methyl-D-alanine, N-methyl-D-valine, N-methyl-D-leucine, N-methyl-D-isoleucine, N-methyl-D-proline, N-methyl-D-methionine, N-methyl-D-phenylalanine, N-methyl-D-tryptophan, N-methyl-D-γ-aminoisobutyric acid, N-methyl-L-cyclohexylalanine, N-methyl-D-cyclohexylalanine, N-methyl-L-cyclopentylalanine, N-methyl-D-cyclopentylalanine, N-methyl-L-norleucine, N-methyl-D-norleucine, N-methyl-L-norvaline, N-methyl-D-norvaline, N-methyl-L-tent-butylglycine, N-methyl-D-tert-butylglycine, N-methyl-L-ethylglycine and N-methyl-D-ethylglycine.

As used herein, the term “positively charged amino acid residue” refers to an amino acid residue having a side chain capable of bearing a positive charge. Examples include, but are not limited to L-lysine, L-arginine, L-histidine, L-ornithine, D-lysine, D-arginine, D-histidine, D-ornithine, N-methyl-L-lysine, N-methyl-L-arginine, N-methyl-L-histidine, N-methyl-L-ornithine, N-methyl-D-lysine, N-methyl-D-arginine, N-methyl-D-histidine, N-methyl-D-ornithine, L-diaminobutyric acid (DAB), D-diaminobutyric acid, N-methyl-L-diaminobutyric acid, N-methyl-D-diaminobutyric acid, L-citrulline (CIT), D-citrulline, N-methyl-L-citrulline, N-methyl-D-citrulline, L-homoarginine, D-homoarginine, N-methyl-L-homoarginine and N-methyl-D-homoarginine.

As used herein, the term “negatively charged amino acid residue” refers to an amino acid residue having a side chain capable of bearing a negative charge. Examples include, but are not limited to L-aspartic acid, L-glutamic acid, D-aspartic acid, D-glutamic acid, N-methyl-L-aspartic acid, N-methyl-L-glutamic acid, N-methyl-D-aspartic acid and N-methyl-D-glutamic acid.

As used herein, the term “polar uncharged amino acid residue” refers to an amino acid residue having a side chain that is uncharged and has a dipole moment. Examples of polar amino acid residues, include, but are not limited to glycine, sarcosine, L-serine, L-threonine, L-cysteine, L-tyrosine, L-asparagine, L-glutamine, D-serine, D-threonine, D-cysteine, D-tyrosine, D-asparagine and D-glutamine, N-methyl-L-serine, N-methyl-L-threonine, N-methyl-L-cysteine, N-methyl-L-tyrosine, N-methyl-L-asparagine, N-methyl-L-glutamine, N-methyl-D-serine, N-methyl-D-threonine, N-methyl-D-cysteine, N-methyl-D-tyrosine, N-methyl-D-asparagine, N-methyl-D-glutamine, L-homoarginine, D-homoarginine, N-methyl-L-homoarginine, N-methyl-D-homoarginine, L-O-methyltyrosine, D-O-methyltyrosine, N-methyl-L-O-methyltyrosine, N-methyl-D-O-methyltyrosine, L-homotyrosine, D-homotyrosine, N-methyl-L-homotyrosine, N-methyl-D-homotyrosine, L-O-methylhomotyrosine, D-O-methylhomotyrosine, N-methyl-L-O-methylhomotyrosine and N-methyl-D-O-methylhomotyrosine.

The term “amino acid having a small side chain” refers to amino acid residues having a side chain with 4 or less non-hydrogen atoms, especially 3 or less non-hydrogen atoms. Examples include, but are not limited to, glycine, sarcosine, L-alanine, L-valine, L-leucine, L-isoleucine, L-methionine, L-serine, L-threonine, L-cysteine, L-asparagine, L-aspartic acid, L-ethylglycine, L-tent-butylglyeine, D-alanine, D-valine, D-leucine, D-isoleucine, D-methionine, D-serine, D-threonine, D-cysteine, D-asparagine, D-aspartic acid, D-ethylglycine and D-tent-butylglyeine, especially glycine, L-alanine, L-valine, L-serine, L-threonine, L-cysteine, L-alanine, L-valine, L-serine, L-threonine and L-cysteine.

The term “conservative amino acid substitution” refers to substituting one amino acid in a sequence with another amino acid that has similar properties of size, polarity and/or aromaticity and does not change the nature or activity of the peptide. For example, one polar amino acid residue may be substituted with another polar amino acid residue or an amino acid residue having a small side chain may be substituted with another amino acid residue having a small side chain.

The compounds of the invention may be in the form of pharmaceutically acceptable salts. It will be appreciated, however, that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quaternised with such agents as lower alkyl halide, such as methyl, ethyl, propyl, and butyl chlorides, bromides and iodides; dialkyl sulfates like dimethyl and diethyl sulfate; and others.

It will also be recognised that compounds of the invention may possess asymmetric centres and are therefore capable of existing in more than one stereoisomeric form. The invention thus also relates to compounds in substantially pure isomeric form at one or more asymmetric centres e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution. The compounds of the invention may exist as geometric isomers. The invention also relates to compounds in substantially pure cis (Z) or trans (E) or mixtures thereof.

The compounds of the invention may also be in the form of solvates, including hydrates. The term “solvate” is used herein to refer to a complex of variable stoichiometry formed by a solute (a compound of formula (I)) and a solvent. Such solvents should not interfere with the biological activity of the solute. Solvents that may be included in a solvate include, but are not limited to, water, ethanol, propanol, and acetic acid. Methods of solvation are generally known within the art.

The term “pro-drug” is used in its broadest sense and encompasses those derivatives that are converted in vivo to the compounds of formula (I). Such derivatives would readily occur to those skilled in the art and include, for example, compounds where a free hydroxy group is converted into an ester derivative or a free nitrogen is converted to an N-oxide. Examples of ester derivatives include alkyl esters, phosphate esters and those formed from amino acids. Conventional procedures for the preparation of suitable prodrugs are described in text books such as “Design of Prodrugs” Ed. H. Bundgaard, Elsevier, 1985.

### COMPOUNDS OF THE INVENTION

In one aspect of the present invention there is provided a compound of formula (I):

R₁-Xaa₁-Pro-c(Xaa₂-Xaa₃-Xaa₄-Xaa₅-Xaa₆-Xaa₇-Xaa₈-Xaa₉)-Xaa₁₀-R₂ (I)
wherein
R₁ is acyl or hydrogen;
R₂ is OH or NH₂;
Xaa₁ is a hydrophobic amino acid residue,
Xaa₂ is selected from SSA, Asp, Glu and Cys;
Xaa₃ is an amino acid residue having a small side chain;
Xaa₄ is a hydrophobic amino acid residue;
Xaa₅ is a polar uncharged amino acid residue;
Xaa₆ is a negatively charged amino acid residue;
Xaa₇ and Xaa₈ are each independently absent or an amino acid with a small side chain;
Xaa₉ is selected from SSA, Asp, Glu and Cys; and
Xaa₁₀ is a positively charged amino acid residue;
wherein one of the following applies:
   iv) Xaa₂ is SSA and Xaa₉ is Asp or Glu;
   v) Xaa₂ is Asp or Glu and Xaa₉ is SSA; or
   vi) Xaa₂ and Xaa₉ are both Cys;
wherein Xaa₂ and Xaa₉ are linked to form a cyclic peptide; and wherein SSA is an amino acid residue of formula (II):

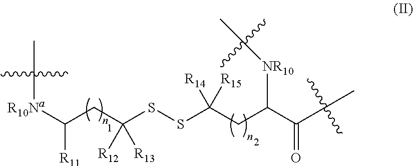

wherein
each R₁₀ is independently selected from hydrogen, substituted or unsubstituted C₁₋₆ alkyl;
R₁₁ is selected from hydrogen and methyl;
R₁₂, R₁₃, R₁₄ and R₁₅ are each independently selected from the group consisting of hydrogen and C₁₋₄ alkyl, or R₁₂ and R₁₃ taken together with the carbon atom to which they are attached and/or R₁₄ and R₁₅ taken together with the carbon atom to which they are attached for a cyclic structure selected from C₃₋₅ cycloalkyl and C₃₋₅ oxygen-containing, nitrogen containing or sulfur-containing heterocycle, each of which may be substituted or unsubstituted; and
n₁ and n₂ are each independently 0, 1, 2, 3 or 4;
with the proviso that at least one of R₁₂, R₁₃, R₁₄ and R₁₆ is not hydrogen;
wherein SSA is linked to Xaa₂ or Xaa₉ via N^{a};
or a pharmaceutically acceptable salt thereof.

In some embodiments Xaa₂ is SSA and Xaa₉ is Asp or Glu, especially where Xaa₉ is Glu.

In some embodiments, Xaa₂ is Asp or Glu and Xaa₉ is SSA, especially where Xaa₂ is Asp.

In some embodiments, Xaa₂ and Xaa₉ are both Cys and are linked to form a disulfide bond.

In some embodiments, SSA is an amino acid residue of formula (III) or formula (IV):

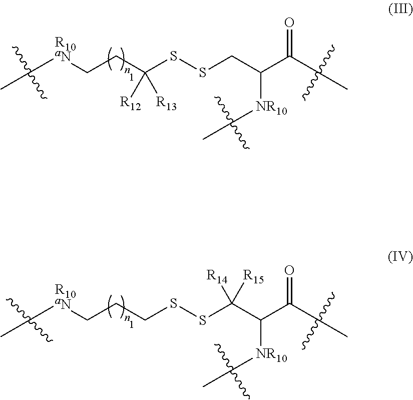

where R₁₀, R₁₁, R₁₂, R₁₃, R₁₄, R₁₅ and n₁ are as defined in formula (I).

In some embodiments, one or more of the following applies in relation to formula (II):
i) Each R₁₀ is independently selected from hydrogen and methyl, especially where one or both R₁₀ are hydrogen;
ii) R₁₁ is hydrogen;
iii) Each R₁₂, R₁₃, R₁₄, R₁₅ are independently selected from hydrogen and C₁₋₃ alkyl, especially hydrogen and methyl; especially where R₁₂ and R₁₃ are both methyl and R₁₄ and R₁₅ are both hydrogen or where R₁₂ and R₁₃ are both hydrogen and R₁₄ and R₁₅ are both methyl; especially where R₁₂ and R₁₃ are both methyl and R₁₄ and R₁₅ are both hydrogen;
iv) n₁ and n₂ are independently 0, 1 or 2, especially 0 or 1, more especially 0.

In particular embodiments, SSA is an amino acid residue of the formula (V):

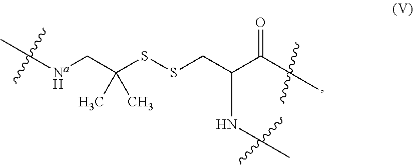

(also referred to as SSA* in the sequences of the invention). This amino acid is S-((1-amino-2-methylpropan-2-yl)thio)-L-cysteine, when made from L-cysteine and S-((1-amino-2-methylpropan-2-yl)thio)-D-cysteine, when made from D-cysteine.

In particular embodiments, one or more of the following applies in relation to formula (I):
i) R₁ is hydrogen;
ii) R₂ is NH₂;
iii) Xaa₁ is leucine, isoleucine, alanine or t-butylglycine; especially leucine or t-butylglycine;
iv) Xaa₃ is alanine;
v) Xaa₄ is leucine, isoleucine, alanine or t-butylglycine; especially leucine or t-butylglycine;
vi) Xaa₅ is asparagine or glutamine; especially asparagine;
vii) Xaa₆ is aspartic acid or glutamic acid, especially aspartic acid;
viii) Xaa₇ is absent or is glycine, especially absent;
ix) Xaa₈ is absent or is glycine, especially absent;
x) Xaa₁₀ is lysine or arginine, especially arginine.

In particular embodiments, the compound of formula (I) is selected from:

SEQ ID NO: 1
Leu-Pro-c(SSA*-Ala-Leu-Asn-Asp-Glu)-Arg-NH₂
SEQ ID NO: 2
Leu-Pro-c(SSA*-Ala-Leu-Asn-Asp-Glu)-Lys-NH₂
SEQ ID NO: 3
Leu-Pro-c(SSA*-Ala-Leu-Asn-Asp-Asp)-Lys-NH₂
SEQ ID NO: 4
Leu-Pro-c(SSA*-Ala-tBG-Asn-Asp-Glu)-Lys-NH₂
SEQ ID NO: 5
Leu-Pro-c(SSA*-Ala-tBG-Asn-Asp-Glu)-Arg-NH₂
SEQ ID NO: 6
tBG-Pro-c(SSA*-Ala-tBG-Asn-Asp-Glu)-Lys-NH₂
SEQ ID NO: 7
tBG-Pro-c(SSA*-Ala-tBG-Asn-Asp-Glu)-Arg-NH₂
SEQ ID NO: 8
tBG-Pro-c(SSA*-Ala-Leu-Asn-Asp-Glu)-Lys-NH₂
SEQ ID NO: 9
tBG-Pro-c(SSA*-Ala-Leu-Asn-Asp-Glu)-Arg-NH₂
SEQ ID NO: 10
Leu-Pro-c(SSA*-Ala-Leu-Asn-Asp-Gly-Gly-Glu)-Lys-
NH₂
SEQ ID NO: 11
Leu-Pro-c(SSA*-Ala-Leu-Asn-Asp-Gly-Gly-Glu)-Arg-
NH₂
SEQ ID NO: 12
Leu-Pro-c(SSA*-Ala-tBG-Asn-Asp-Gly-Gly-Glu)-Lys-
NH₂
SEQ ID NO: 13
Leu-Pro-c(SSA*-Ala-tBG-Asn-Asp-Gly-Gly-Glu)-Arg-
NH₂
SEQ ID NO: 14
tBG-Pro-c(SSA*-Ala-tBG-Asn-Asp-Gly-Gly-Glu)-Lys-
NH₂
SEQ ID NO: 15
tBG-Pro-c(SSA*-Ala-tBG-Asn-Asp-Gly-Gly-Glu)-Arg-
NH₂
SEQ ID NO: 16
tBG-Pro-c(SSA*-Ala-Leu-Asn-Asp-Gly-Gly-Glu)-Lys-
NH₂
SEQ ID NO: 17
tBG-Pro-c(SSA*-Ala-Leu-Asn-Asp-Gly-Gly-Glu)-Arg-
NH₂
SEQ ID NO: 18
Leu-Pro-c(SSA*-Ala-Leu-Asn-Asp-Gly-Gly-Asp)-Lys-
NH₂
SEQ ID NO: 19
Leu-Pro-c(SSA*-Ala-Leu-Gln-Asp-Gly-Gly-Asp)-Lys-
NH₂
SEQ ID NO: 20
Leu-Pro-c(SSA*-Gly-Leu-Asn-Asp-Gly-Gly-Asp)-Lys-
NH₂
SEQ ID NO: 21
Leu-Pro-c(SSA*-Ala-tBG-Asn-Asp-Gly-Gly-Asp)-Lys-
NH₂
SEQ ID NO: 22
Leu-Pro-c(SSA*-Ala-Leu-Gln-Asp-Gly-Gly-Asp)-Lys-
NH₂
SEQ ID NO: 23
Leu-Pro-c(Asp-Ala-Leu-Asn-Asp-SSA*)-Lys-NH₂
SEQ ID NO: 24
Ile-Pro-c(Asp-Ala-Leu-Asn-Asp-SSA*)-Arg-NH₂
SEQ ID NO: 25
Leu-Pro-c(Asp-Ala-Leu-Asn-Asp-SSA*)-Arg-NH₂
SEQ ID NO: 26
Ile-Pro-c(Asp-Ala-Ile-Asn-Asp-SSA*)-Arg-NH₂
SEQ ID NO: 27
Leu-Pro-c(Asp-Ala-Ile-Asn-Asp-SSA*)-Arg-NH₂
SEQ ID NO: 28
Leu-Pro-c(Asp-Ala-Leu-Asn-Glu-SSA*)-Arg-NH₂
SEQ ID NO: 29
Leu-Pro-c(Cys-Ala-Leu-Asn-Asp-Gly-Gly-Cys)-Lys-
NH₂
SEQ ID NO: 30
Leu-Pro-c(Cys-Ala-Leu-Asn-Asp-Gly-Gly-Cys)-Lys-
COOH.

The amino acid SSA of formula (I) may be obtained by synthesis as set out in WO2019/018892. Other amino acids may be purchased. Synthesis of the peptides of the invention may be performed by methods known in the art such as solid phase or solution phase synthesis using a protecting group strategy such as Fmoc or Boc chemistry.

In a particular embodiment, the compound of formula (I) is SEQ ID NO:1.

According to another aspect of the invention there is provided a pharmaceutical composition comprising a compound of formula (I) or a pharmaceutically acceptable salt, stereoisomer or prodrug thereof, and a pharmaceutically acceptable carrier, diluent and/or excipient.

Suitably, the pharmaceutically acceptable carrier, diluent and/or excipient may be or include one or more of diluents, solvents, pH buffers, binders, fillers, emulsifiers, disintegrants, polymers, lubricants, oils, fats, waxes, coatings, viscosity-modifying agents, glidants and the like.

The salt forms of the compounds of the invention may be especially useful due to improved solubility.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Disintegrants may include one or more of starch, croscarmellose sodium, crospovidone, sodium starch glycolate and the like. Solvents may include one or more of ethanol, methanol, isopropanol, chloroform, acetone, methylethyl ketone, methylene chloride, water and the like. Lubricants may include one or more of magnesium stearate, zinc stearate, calcium stearate, stearic acid, sodium stearyl fumarate, hydrogenated vegetable oil, glyceryl behenate and the like. A glidant may be one or more of colloidal silicon dioxide, talc or cornstarch and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

Reference is made to the Handbook of Excipients 6th Edition, Eds. Rowe, Sheskey & Quinn (Pharmaceutical Press), which provides non-limiting examples of excipients which may be useful according to the invention.

It will be appreciated that the choice of pharmaceutically acceptable carriers, diluents and/or excipients will, at least in part, be dependent upon the mode of administration of the formulation. By way of example only, the composition may be in the form of a tablet, capsule, caplet, powder, an injectable liquid, a suppository, a slow release formulation, an osmotic pump formulation or any other form that is effective and safe for administration. In particular embodiments, where the cancer being treated is brain cancer, the compound of formula (I) may be delivered directly to the brain, for example using nose to brain delivery via a sol-gel nasal formulation or using an intra-cerebroventricular delivery system.

### METHODS OF TREATMENT

The compounds of the present invention are suitable for the treatment of cancers in which matrix metalloproteinases (MMPs) or other proteinases such as urokinase-type plasminogen activator (uPa) are implicated. MMPs are known to exert effects on the extracellular microenvironment, for example, degradation of the extracellular matrix, thereby allowing cancer cell invasion. Such cancers include malignant ascites such as pancreatic cancer, colorectal cancer, gastric cancer, ovarian cancer, cholangiocarcinoma and mesothelioma; malignant pleural effusion such as non-small cell lung cancer, breast cancer, renal cancer, melanoma, and mesothelioma; prostate cancer; small cell lung cancer; esophageal cancer; fibrosarcoma and central nervous system cancers such as brain cancers.

In particular embodiments, the compounds of the present invention are for the treatment or prevention of central nervous system cancer. In some embodiments, the central nervous system cancer is brain cancer. In other embodiments, the central nervous system cancer is spinal cord cancer. In some embodiments, the central nervous system cancer is glioma, especially glioblastoma. In particular embodiments, the compounds are for the treatment of glioblastoma multiforme. In some embodiments, the brain cancer is a medulloblastoma. In particular embodiments, the central nervous system cancer is brain cancer.

Without wishing to be bound by theory, the invasive growth of cancers such as medulloblastoma and glioblastoma tumors, (for example, glioblastoma multiforme), relies strongly on the restructuring of the extracellular matrix (ECM). ECM restructuring is induced by the serine protease urokinase plasminogen activator (uPA) and is carried out by plasmin and matrix metalloproteases such as MMP-2 and MMP-9. uPA plays a prominent role in activation of plasmin and MMPs and therefore the degradation of ECM (Schuler et al. 2012).

In some embodiments, the glioma is low grade glioma. In some embodiments the glioma is associated with over expression of or increased activity of uPA and/or matrix metalloproteinases, especially MMP-2 and/or MMP-9.

In another aspect of the present invention, there is provided a method of inhibiting urokinase plasminogen activator (uPA) and/or a matrix metalloproteinase comprising contacting the matrix metalloproteinase with a compound of formula (I) or a pharmaceutically acceptable salt thereof. In some embodiments, uPA activity is inhibited. In some embodiments, the matrix metalloproteinase is MMP-2. In some embodiments, the matrix metalloproteinase is MMP-9. In some embodiments, the matrix metalloproteinase is MMP-2 and MMP-9. In some embodiments, the uPA and/or matrix metalloproteinase is in vivo. In other embodiments, the uPA and/or matrix metalloproteinase is in vitro.

In some embodiments, the methods of the invention inhibit breakdown of the brain extracellular matrix, thereby reducing invasiveness of the brain tumour. In another aspect of the present invention there is provided a method of inhibiting tumour cell invasion in a brain tumour comprising administering to the brain tumour with a compound of formula (I) according to the invention or a pharmaceutically acceptable salt thereof.

As generally used herein, the terms “administering” or “administration”, and the like, describe the introduction of the compound or composition to a subject such as by a particular route or vehicle. Routes of administration may include topical, parenteral and enteral which include oral, buccal, sub-lingual, nasal, anal, gastrointestinal, subcutaneous, intramuscular, intravenous, intrathecal, intracranial, intra-arterial, intraventricular and intradermal routes of administration, although without limitation thereto.

By “treat”, “treatment” or treating” is meant administration of the compound or composition to a subject to at least alleviate, reduce or suppress the central nervous system cancer in the subject. Treatment does not mean that the cancer is cured completely, treating also includes halting progression of a tumour, reducing the size of a tumour or alleviating the symptoms of a tumour.

By the term “inhibiting” is meant that the compound of formula (I) blocks the activity of a metalloproteinase enzyme or reduces the rate of activity of a metalloproteinase enzyme.

An “effective amount” means an amount necessary at least partly to attain the desired response, or to alleviate, decrease or remove the pain, delay the onset or inhibit progression of the pain, or inhibit the onset of the pain being treated. The amount varies depending upon the health and physical condition of the individual to be treated, the taxonomic group of individual to be treated, the degree of alleviation desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage. Dosage regimes may be adjusted to provide the optimum therapeutic response. For example, several divided doses may be administered daily, weekly, monthly or other suitable time intervals, or the dose may be proportionally reduced as indicated by the exigencies of the situation. The effective amount and appropriate dosage regimen may be ascertained through routine trial.

As used herein, the terms “subject” or “individual” or “patient” may refer to any subject, particularly a vertebrate subject, and even more particularly a mammalian subject, for whom treatment is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). In a particular embodiment, the subject is a human.

In another aspect of the present invention, there is provided a use of a compound of formula (I) or a pharmaceutically acceptable salt thereof in the manufacture of a medicament for treating or preventing a cancer. In particular embodiments, the cancer is a central nervous system cancer.

In yet another aspect, there is provided a compound of formula (I) or a pharmaceutically acceptable salt thereof for use in treating or preventing a cancer. In particular embodiments, the cancer is a central nervous system cancer.

In yet another aspect of the present invention, there is provided a use of a compound of formula (I) or a pharmaceutically acceptable salt thereof in the manufacture of a medicament for inhibiting tumour cell invasion in a brain tumour.

In a further aspect of the present invention, there is provided a compound of formula (I) or a pharmaceutically acceptable salt thereof for use in inhibiting tumour cell invasion in a brain tumour.

In some embodiments, the compound of formula (I) is administered in combination with other treatments for central nervous system cancers such as brain cancers. For example, the compounds of formula (I) may be administered in a single composition or in separate compositions simultaneously or sequentially, with a CDK4/6 inhibitor such as palbociclib or a microtubule stabilizer such as Ixabepilone or taxol or a chemotherapy selected from Temozolomide, Savolitinib, Terameprocol, Ivosidenib, Veliparib or Abemaciclib. In some embodiments, the combination is given in combination with radiation therapy, for example, a compound of formula (I), Temozolomide and radiation therapy.

### EXAMPLES

### Example 1: In Silico Docking Studies

Designed cyclic peptides were docked using the Glide module of the Schrodinger Suite 11.5 to determine their binding mode/interactions. The crystal structure of human MMP-2 (PDB Code 3AYU) with a bound decapeptide ligand was used in the modelling studies (EP2623111A2). A protein file compatible for the Glide docking was prepared by adding the hydrogens, converting the selenomethionines to methionines, adding the missing residues and side-chains. The whole protein was minimized using the OPLS 2005 force field. The minimized protein structure was used to generate a search grid (a 3-dimensional search box of a defined length encompassing the active site residues) by selecting the centroid of the co-crystal bound peptide. The search volume (distance between the centroid of the selected inhibitor and the faces of the search grid cube) was limited to 16 Å, which was sufficient to accommodate the designed peptides. All peptide molecules were prepared for docking by using the Ligprep module of the Schrodinger software. All possible ionization states and metal binding states were generated for pH 7 using the Epic module of the Schrödinger Maestro. Flexible and standard precision peptide (SP-peptide) docking was performed by allowing the side chain hydroxyl groups to rotate around the bond axis. This generated all possible binding conformations within the binding site. The docking gave affinity scores known as Glide Scores (referred to as docking scores) calculated by Equation (1) below:

Glide Score=0.65*vdW+0.130*Coul+Lipo+HBond+Metal+BuryP+RotB+Site (1)

Where:
vdW is Van der Waals energy,
Coul is Coulomb energy
Lipo is lipophilic term derived from hydrophobic grid potential
HBond is hydrogen-bonding term;
Metal is metal-binding term;
BuryP is penalty for buried polar groups;
RotB is penalty for freezing rotatable bonds; and
Site is polar interactions in the active site.

To perform docking, energy in rotational and translational degrees of freedom was first minimized. Binding energy calculations and complementarity scores were used for the evaluation of docking. The negative an low value of binding energy showed strong and most favourable binding between protein (MMP-2) and ligand (designed peptide) molecules. Moreover, the complex strength is influenced by the number of H2 bonds and electrostatic energy. The binding score and binding energies of peptides with MMP-are shown in Table 3.

TABLE 3

| | | Docking | Binding |
|---|---|---|---|
| Compound | Peptide Sequence | Score | Energy |
| SEQ ID NO: 1 | Leu-Pro-c(SSA*-Ala-Leu-Asn-Asp-Glu)- | −8.60 | −20.06 |
| | Arg-NH₂ | | |
| SEQ ID NO: 2 | Leu-Pro-c(SSA*-Ala-Leu-Asn-Asp-Glu)- | −5.573 | −10.062 |
| | Lys-NH₂ | | |
| SEQ ID NO: 3 | Leu-Pro-c(SSA*-Ala-Leu-Asn-Asp-Asp)- | −6.019 | −10.461 |
| | Lys-NH₂ | | |
| SEQ ID NO: 4 | Leu-Pro-c(SSA*-Ala-tBG-Asn-Asp-Glu)- | −9.08 | −13.902 |
| | Lys-NH₂ | | |
| SEQ ID NO: 5 | Leu-Pro-c(SSA*-Ala-tBG-Asn-Asp-Glu)- | −5.991 | −16.152 |
| | Arg-NH₂ | | |
| SEQ ID NO: 6 | tBG-Pro-c(SSA*-Ala-tBG-Asn-Asp-Glu)- | −4.666 | −21.071 |
| | Lys-NH₂ | | |
| SEQ ID NO: 7 | tBG-Pro-c(SSA*-Ala-tBG-Asn-Asp-Glu)- | −4.801 | −3.451 |
| | Arg-NH₂ | | |
| SEQ ID NO: 8 | tBG-Pro-c(SSA*-Ala-Leu-Asn-Asp-Glu)- | −3.71 | −0.972 |
| | Lys-NH₂ | | |
| SEQ ID NO: 9 | tBG-Pro-c(SSA*-Ala-Leu-Asn-Asp-Glu)- | −7.366 | −7.272 |
| | Arg-NH₂ | | |
| SEQ ID NO: 12 | Leu-Pro-c(SSA*-Ala-tBG-Asn-Asp-Gly- | −6.2 | −10.73 |
| | Gly-Glu)-Lys-NH₂ | | |
| SEQ ID NO: 14 | tBG-Pro-c(SSA*-Ala-tBG-Asn-Asp-Gly- | −6.554 | 0.742 |
| | Gly-Glu)-Lys-NH₂ | | |
| SEQ ID NO: 23 | Leu-Pro-c(Asp-Ala-Leu-Asn-Asp-SSA*)- | −8.193 | −7.508 |
| | Lys-NH₂ | | |
| SEQ ID NO: 24 | Ile-Pro-c(Asp-Ala-Leu-Asn-Asp-SSA*)- | −5.528 | −0.286 |
| | Arg-NH₂ | | |
| SEQ ID NO: 25 | Leu-Pro-c(Asp-Ala-Leu-Asn-Asp-SSA*)- | 6.523 | −21.431 |
| | Arg-NH₂ | | |
| SEQ ID NO: 26 | Ile-Pro-c(Asp-Ala-Ile-Asn-Asp-SSA*)- | −7.167 | −5.242 |
| | Arg-NH₂ | | |
| SEQ ID NO: 27 | Leu-Pro-c(Asp-Ala-Ile-Asn-Asp-SSA*)- | −8.377 | −0.034 |
| | Arg-NH₂ | | |
| SEQ ID NO: 28 | Leu-Pro-c(Asp-Ala-Leu-Asn-Glu-SSA*)- | −4.829 | −18.277 |
| | Arg-NH₂ | | |
| SEQ ID NO: 29 | Leu-Pro-c(Cys-Ala-Leu-Asn-Asp-Gly- | −9.078 | −14.436 |
| | Gly-Cys)-Lys-NH₂ | | |
| SEQ ID NO: 30 | Leu-Pro-c(Cys-Ala-Leu-Asn-Asp-Gly- | −7.021 | −16.511 |
| | Gly-Cys)-Lys-COOH | | |

SEQ ID NO: 1 was selected for synthesis and in vitro investigation.

### Example 2: Peptide Synthesis

SSA* was prepared synthetically as set out in WO2019/018892.

The peptide of SEQ ID NO. 1 was synthesized using solid phase synthesis using standard Fmoc Chemistry. Rink amide resin, Fmoc-amino acids and Oxyma Pure were purchased from Chem-Impex International USA. N,N-dimethylformamide (DMF), dichloromethane (DCM), acetonitrile, N,N′-diisopropylcarobdiimide (DIPCDI), diethyl ether, triisopropylsilane (TIPS), formic acid, trifluoroacetic acid (TFA), piperidine, sodium sulfate, tetrahydrofuran (THF) were purchased from Sigma Aldrich, Australia.

The peptide was synthesized using a Biotage® Initiator+Alstra™ instrument. Standard Fmoc solid phase synthesis was used. The synthesis was carried out on Rink amide resin (0.47 meq/g). All required Fmoc-amino acids were carefully weighed into 25 mL vials, followed by dissolution into the recommended amount of DMF solvent. Oxyma Pure and DIPCDI were used for sequential coupling of amino acids and all coupling reactions were performed under microwave conditions except for Asp, Glu and Arg residues, which were performed at room temperature. Selective deprotection of SSA and Glu side chains (ODmab & Dde) which were orthogonal to Fmoc, was performed using hydroxylamine hydrochloride imidazole (1.3:1) in N-methyl pyrrolidine (NMP). Cyclisation of the deprotected SSA and Glu was performed in the same manner as coupling of the amino acids using Oxyma Pure and DIPCDI. Fmoc deprotection was performed using 20% v/v piperidine in DMF. To prevent aspartamide formation 1% formic acid in 20% v/v piperidine was used for deprotection of Asp. After completion of the synthesis, the dry resin was collected from the synthesizer and off-resin cleavage performed using the cleavage cocktail (TFA:TIPS:H₂O:DCM, 90:2.5:2.5:5). The crude peptide was collected and purified by preparative HPLC.

Preparative HPLC was performed using an Agilent 1200 Chem station equipped with a binary pump and auto-fraction collector. A Jupiter 10 μm Proteo 90 Å LC column 250×21.2 mm was used with a flow rate of 10 mL/min. The mobile phase employed was solvent A: MilliQ water, solvent B: acetonitrile, both containing 0.1% v/v TFA with a gradient flow 5% to 100% B for 30 minutes.

The peptide was characterized by ESI-MS in a solution of water:acetonitrile (1:1) and a concentration of 100 μg/mL prior to direct injection into an LC/MS/MS (ABSciex API 2000™), positive ion mode with declustering potential (DP) and entrance potential (EP) set at 200 and 10 mV respectively.

The results are provided in Table 4:

TABLE 4

| | No. of | | | Observed | HPLC |
|---|---|---|---|---|---|
| | Amino | | Calculated | m/z | purity |
| Compound | acids | Sequence | m/z | [M + H]⁺ | % |
| SEQ ID NO: 1 | 9 | LPc(XALNDE)R- | 1113.5 | 1114.2 | 99.7 |
| | | NH2 | | | |

### Example 3: MTT Assay for Cytotoxicity

The MTT (3-[4.5-dimethylthiazol-2-yl]-2,5═diphenyl tetrazolium bromide) assay is based on a process undertaken by living cells, involving the conversion of MTT into formazan crystals. This assay is used routinely to measure the in vitro cytotoxic effects of drugs on cell lines or primary patient cells (Van Meerloo et al., 2011). The MTT assay measures mitochondrial activity, so in some instances where the peptide activates mitochondrial response, the results appear to increase the number of cells present i.e. >100% cell viability.

The aim of this experiment was to identify peptide concentrations that are active on living cells and cell viability was assessed.

The glioblastoma multiforme (GBM) cell lines used were U87 and U251 cells and patient derived cells grown as oncospheres (081024 cells). Human adherent GBM cell lines U87 and U251 were cultured in RPMI medium (Life Technologies Melbourne Australia) supplemented with 5% v/v FBS, 100 U/mL penicillin and 100 μg/mL streptomycin. 081024 cells were cultrured in NeuroCult™ NS-A Proliferation medium with 0.2% (v/v) heparin, 20 ng/mL EGF and 10 ng/mL FGF.

8×10³ U87 and 7×10³ U251 cells per well were seeded in 96 well plates in 200 μL serum-containing RPMI medium and incubated at 37° C. with 5% CO₂ for 24 hours. After one wash with serum-free medium, 100 μL serum-free medium with different concentration of peptide SEQ ID NO:1 was added to each well. After 48 hours incubation, 10 μL of 0.5 mg/mL MTT was added and incubated for 2 hours at 37° C. Formazan crystals were dissolved by 200 μL/well dimethyl sulfoxide (DMSO) and shaking for 15 minutes. The UV absorbance was read with an iMark™ microplate absorbance reader at 595 nm (Bio-Rad Laboratories). Background absorbance was subtracted and results expressed as the % viability of control (untreated cells). The results are shown in Table 5.

TABLE 5
Cell viability at different concentrations of SEQ ID NO: 1

| Cell | 1 | 5 | 10 | 50 | 100 | 500 | 1 |
|---|---|---|---|---|---|---|---|
| Line | μM | μM | μM | μM | μM | μM | mM |
| U87 | 96.34 ± | 102.4 ± | 100.1 ± | 98.71 ± | 102.8 ± | 96.94 ± | 83.41 ± |
| | 10.7 | 24.7 | 15.1 | 13.9 | 11.8 | 9.1 | 14.6 |
| U251 | 100.7 ± | 101.7 ± | 100.4 ± | 107.3 ± | 105.5 ± | 93.84 ± | 83.99 ± |
| | 12.3 | 10.0 | 4.8 | 4.2 | 4.2 | 7.5 | 10.0 |
| 081024 | 104.2 ± | 106.8 ± | 106.7 ± | 106.5 ± | 106.9 ± | 99.68 ± | 89.58 ± |
| | 3.5 | 4.6 | 2.6 | 3.3 | 0.3 | 2.4 | 3.7 |

In light of the results, a peptide concentration of 0.5 mM (500 μM) was chosen for subsequent in vitro studies, this being the highest concentration which did not significantly affect cell viability.

### Example 4: Effects of Peptides on Production of Matrix Proteinases

To assess whether the peptide SEQ ID NO.1 impacted MMP-2, MMP-9 and uPA production Zymography was used.

Zymography is a technique for studying hydrolytic enzymes on the basis of substrate degradation [23]. The in-gel zymography hydrolytic enzymes are separated by their molecular weights and detected by their ability to degrade a substrate. To detect the activity of MMP-2 and MMP-9 in various cell lines, conditioned media were collected in 3 independent experiments and analysed by gelatin zymography to quantify the level of gelatin digestion, which reflects MMP-2 and MMP-9 activity and production. Casein-Plasminogen zymography was conducted to determine the activity level of uPA in the conditioned medium from various cell lines. Active uPA converts plasminogen into plasmin which then digests casein, thus clear bands where digestion of casein have occurred indirectly represent uPA activity.

The effect of the peptides on matrix protease production was tested using in-gel zymography. If the substrate included in the gel is gelatin, the enzymes detected are gelatinases: MMP-2 and MMP-9 (the latter is often of low abundance). If the gel contains casein and plasminogen, the enzymes tested are urokinase plasminogen activators (uPA); uPA is a therapeutic target in GBM.

An equal amount of protein from each sample was mixed with 5 μL of Laemmli buffer (0.01% Bromophenol Blue (W/V), 5% SDS (W/V), 22% glycerol (V/V), 14% 0.5 M Tris pH 6.8 (V/V)) and loaded onto a 10% polyacrylamide gel co-polymerized with 1 mg/ml of gelatin, and electrophoresed at 120 V for 2 hours on ice. The gel was incubated in denaturing solution (Triton X-100 2.5% (V/V), 1 M Tris pH 7.5 5% (V/V), 0.5 M CaCl₂ 1% (W/V) at room temperature overnight on an orbital shaker. The gel was incubated in a second solution for 3 hours at 37° C. The gel was then stained with Coomassie blue solution (Coomassie blue 0.25%, methanol 45%, acetic acid 10%) and destained in methanol 25% (V/V), acetic acid 10% (V/V) destaining solution until clear bands against the dark background appeared around 72 kDa and 95 kDa, representing MMP-2 and MMP-9 activity, respectively. Clear bands around 50 kDa appeared against the dark background representing uPA activity. The gels were scanned, and uPA, MMP-2 and MMP-9 were quantified by densitometry using NIH Image J software.

SEQ ID NO.1 was compared to a control containing no peptide to evaluate whether the peptide can reduce the production of matrix proteinases. A reduction in the production of matrix proteinases may be anticipated to result in comparatively less degradation of the extra-cellular matrix and so this could be one pathway by which the invasion potential could be reduced. However, overall, the peptide did not decrease the production of either MMP-2 or uPA. A decreased production of MMP-9 (92 KDa band) was detected, which was apparent in U251 cells, and statistically significant only in the U87 cell line. This could be confirmed using a MMP-9 quantification kit, and altered production by a transcriptional mechanism can be unveiled using qRT-PCR. The peptides did not affect the production of uPA in the cell-conditioned media. The results are shown in Tables 6 and 7.

TABLE 6
metalloprotease production

| | U87 cells | | U251 cells | | 081024 cells | |
|---|---|---|---|---|---|---|
| Peptide | MMP-2 | MMP-9 | MMP-2 | MMP-9 | MMP-2 | MMP-9 |
| SEQ ID | 102.1 ± | 58.61 ± | 110.2 ± | 63.4 ± | 106.4 ± | 98.96 ± |
| NO. 1 | 3.2 | 9.0 | 5.1 | 34.8 | 9.7 | 8.2 |

TABLE 7
uPA production

| Peptide | U87 cells | U251 cells | 081024 cells |
|---|---|---|---|
| SEQ ID NO. 1 | 89.03 ± 5.98 | 79.92 ± 8.86 | 96.43 ± 16.6 |

### Example 5: Effect of Peptide on MMP and UPA Activity

MMP-2 is an important target for inhibitor screening due to its involvement in cancer growth, angiogenesis, and metastasis. The MMP-2 screening assay was carried out with a commercially available MMP-2 Inhibitor Screening Assay Kit (Colorimetric, Catalog: ab139446) based on the manufacturers protocol. This assay is based on quantification of recombinant MMP-2 activity using a colorimetric assay (MMP-2 degrades a chromogenic substrate). The assay provides NNGH (N-isobutyl-N-(4-methoxyphenylsulfonyl) glycyl hydroxamic acid), a small molecule inhibitor, as a positive control (NNGH). This is a potent inhibitor of MMP-3 but it also inhibits other metalloproteinases including MMP-2. Chlorotoxin (commercially sourced control) was also included which is known to inhibit MMP-2. Each determination was run in duplicate. The % inhibition rate was calculated based on the below equation, with a lower % inhibitory rate/activity corresponding to higher inhibitory activity of the peptides.

% Inhibition rate=(Vinhibitor/Vcontrol)×100

Briefly, MMP Substrate and MMP Inhibitor were warmed to room temperature to thaw DMSO. MMP Inhibitor (NNGH), MMP-2 substrate and MMP-2 enzyme were diluted with assay buffer to required concentration. 20 μL of MMP-2 enzyme and MMP-2 inhibitor (peptides, Chlorotoxin and NNGH) was added, then 50 μL of assay buffer was added and the reaction mixture incubated at 37° C. for 1 h. After incubation, 10 μL of MMP Substrate was added to start reaction. The reactions were continuously read at 412 nm in a microplate reader. Data was recorded at 1 min. time intervals for 10 to 20 min. The data as was plotted as optical density (OD) versus time for each sample. The reaction velocity (V) was obtained in OD/min: determine the slope of a line fit to the linear portion of the data plot using an appropriate routine. Then using the equation above the inhibitor % activity remaining was calculated. The control was without inhibitor.

SEQ ID NO.1 showed a dose dependent ability to inhibit MMP-2 activity, (Table 8). Overall the results indicate that the peptide inhibits the activity of MMP-2, and at concentrations that do not affect cell viability.

TABLE 8
MMP-2 inhibitor screening

| | SEQ ID | SEQ ID | SEQ ID | | Chloro- |
|---|---|---|---|---|---|
| | NO. 1 | NO. 1 | NO. 1 | NNGH | toxin |
| Peptide | 1 μM | 10 μM | 100 μM | 1.3 μM | 1 μM |
| % | 81.03 | 75.21 | 66.87 | 2.29 | 67.94 |
| inhibition | | | | | |

### Example 6: Effect of Peptide on mRNA Expression of Invasion-Related Genes

The real-time reverse transcription polymerase chain reaction (RT-PCR) uses fluorescent reporter molecules to monitor the production of amplification products during each cycle of the PCR reaction. This combines the nucleic acid amplification and detection steps into one homogeneous assay and use of appropriate chemistries and data analysis eliminates the need for Southern blotting or DNA sequencing for amplicon identification. Its simplicity, specificity and sensitivity, together with its potential for high throughput and the ongoing introduction of new chemistries, more reliable instrumentation and improved protocols, has made real-time RT-PCR the benchmark technology for the detection and/or comparison of RNA levels.’

In order to detect and quantify the expression of specific genes at mRNA level, real-time reverse transcriptase polymerase chain reaction (Real time RT-PCR) was performed. Total RNA of samples was isolated and purified using the PureLink® RNA Mini Kit (Life technologies). The concentration of total RNA was detected by NanoDrop 2000 Spectrophotometer (Thermo Scientific). The total RNA (2000 ng) was reverse transcribed using the High-Capacity cDNA Reverse Transcription Kit (Applied Biosystems™).

The primers of target genes were TaqMan™ Gene Expression Assay for human PLAU (Hs01547054_m1), MMP-2 (Hs01548727_m1), MMP-9 (Hs00957562_m1), Snail-1 (Hs00195591_m1), Snail-2 (Hs00161904_m1), N-cadherin (Hs00983056_m1) and Twist (Hs01675818_s1). Relative quantification was done by reference to 18S ribosomal RNA (18S rRNA) and analysed using the comparative critical threshold (Ct) method

The mRNA expression of MMPs was measured using qRT-PCR. This was tested three independent times; the results are shown in in Tables 9, 10 and 11 for U251, U87 cells and O81024 cells respectively. Results are reported as percent of the control cells (no treatment). Both MMP-2 and uPA were decreased in U251 by SEQ ID NO.1.

TABLE 9
Effect of peptide SEQ ID NO. 1 on
mRNA expression of U251 cells

| Peptide | uPA | MMP-2 | MMP-9 | Snail-1 | Snail-2 | Twist | CDH-2 |
|---|---|---|---|---|---|---|---|
| SEQ ID | 0.63 ± | 0.72 ± | 0.73 ± | 0.42 ± | 0.75 ± | 0.91 ± | 0.82 ± |
| NO. 1 | 0.04^{a} | 0.1^{a} | 0.19 | 0.17^{a} | 0.06 | 0.06 | 0.08 |

N = 3 independent experiments, the results showed as mean ± SD.
The value with small letters showed significant,
^{a}p < 0.05,
b P < 0.01, one-way ANOVA with Dunnett's multiple comparisons.

TABLE 10
Effect of peptide SEQ ID NO. 1
on mRNA expression of U87cells

| | | MMP- | MMP- | Snail- | Snail- | | CDH- |
|---|---|---|---|---|---|---|---|
| Peptide | uPA | 2 | 9 | 1 | 2 | Twist | 2 |
| SEQ ID | 1.19 ± | 0.84 ± | 0.66 ± | 0.84 ± | 0.94 ± | 0.79 ± | 0.98 ± |
| NO. 1 | 0.42 | 0.27 | 0.25 | 0.26 | 0.13 | 0.13 | 0.35 |

Baseline expression level = ‘1’, with increased expression reflected by values >1, and decreased expression represented by values <1.

TABLE 11
Effect of peptide SEQ ID NO. 1
on mRNA expression of 081024 cells

| Peptide | uPA | MMP-2 | MMP-9 | Snail-1 | Snail-2 | Twist | CDH-2 |
|---|---|---|---|---|---|---|---|
| SEQ ID | 0.85 ± | 0.91 ± | 0.63 ± | 0.60 ± | 1.01 ± | 0.94 ± | 0.91 ± |
| NO. 1 | 0.03 | 0.23 | 0.18 | 0.15^{a} | 0.42 | 0.20 | 0.21 |

Baseline expression level = ‘1’, with increased expression reflected by values >1, and decreased expression represented by values <1.

### Example 7: EMT Marker Expression

Epithelial-mesenchymal transition (EMT) is the process by which epithelial cells acquire a migratory (metastatic) phenotype and so EMT is associated with cancer metastasis, cancer cell stemness, chemoresistance and immune resistance. Therefore, the determination of the mRNA expression of EMT markers is a valuable tool to evaluate invasion potential of cancer cells.

The ‘Snail’ superfamily is involved in cell differentiation and survival, two processes central in cancer research. Snail-1 has a pivotal role in the regulation of epithelial-mesenchymal transition (EMT) and Snail-1 expression is associated with poor prognosis in metastatic cancer, and tumours with elevated Snail-1 expression are harder to treat. The significance of Snail-1 as a prognostic indicator, its involvement in the regulation of EMT and metastasis, and its roles in both drug and immune resistance point out that Snail-1 is an attractive target for tumour growth inhibition and a target for sensitization to cytotoxic drugs (Kaufhold et al., 2014). Twist is transcriptionally active during cell differentiation and lineage determination. During the establishment of cancer metastases by EMT, Twist acts independently of Snail to suppress E-cadherin and to upregulate N-cadherin (CDH-2) and fibronectin. Snail-2 (Slug) is another member of the SNAIL family of transcriptional activators and serves an important role in suppressing the epithelial phenotype in numerous cancer cells (Iwadate, 2016).

SEQ ID NO.1 decreased the expression of the EMT marker Snail-1 in U251 cells and in the oncospheres, where the peptide halved the expression of Snail-1 mRNA with statistical significance. For the CDH-2 and Snail-2, the peptide showed non-statistical decrease in expression. These results are tabulated in Tables 9 to 11 above. Taken together, the peptide reduced expression of EMT markers to some, albeit marginal extent.

### Example 8: Binding/Internalization of the Peptides to GBM Cells

The binding of Chlorotoxin to MMP-2 has been documented and is thought to be the basis for both its anti-invasive action and its ability to specifically label glioma or other cancer cells excluding adjacent non-neoplastic tissue.

To document binding and/or internalization of the peptides to the cells, biotinylated derivatives of the peptides were synthesised to allow detection by Cy3-conjugated avidin staining using confocal fluorescence microscopy.

U87 and U251 cells were seeded on 8-chamber polystyrene vessel tissue culture-treated glass slides (Falcon). After 24 h, cells were incubated with biotinylated peptides (0.5 mM) containing serum free media for 5 min and 6 h. After incubation, the cells were washed with phosphate buffered saline solution (PBS) and fixed with paraformaldehyde 4% (w/v) in PBS for 20 min at room temperature. The cells were rinsed thrice with PBS and permeabilized using PBS containing 0.1% (v/v) Triton X100 over 10 min. After three washes with PBS, cells were incubated with Cy3-streptavidin (3:1000 in PBS) for 10 min. Cells were then rinsed and mounted on microscopic slides with DAPI-containing mounting medium for confocal fluorescence imaging analysis.

All biotin conjugated peptides (biotin conjugated to N-terminal of peptide) were synthesised using a Biotage® Initiator+Alstra™ automated peptide synthesizer. Standard Fmoc solid phase synthesis was used to prepare all peptides. The synthesis was carried out on Rink Amide resin (0.60 meq/g). All required Fmoc-amino acids were carefully weighed into 25 mL vials, followed by dissolution into the recommended amount of DMF solvent. Oxyma Pure and DIPCDI were used for sequential coupling of peptides and all coupling reactions were performed under microwave conditions except for Asp, Glu, SSa and Arg residues, which were performed at room temperature. Fmoc deprotection was performed using 20% v/v piperidine in DMF. To prevent aspartamide formation 1% formic acid in 20% v/v piperidine was used for deprotection of Asp. Separately, for cyclization reaction -Dde and -Odmab orthogonal protecting groups were deprotected using NH₂NH₂·HCl:Imidazole (1.2:1 eq). After completion of synthesis dry resin was collected from the synthesizer and off-resin cleavage performed using an acidic cleavage cocktail (TFA:TIPS:H₂O:DCM, 90:2.5:2.5:5). Crude peptides were collected and further purified by preparative HPLC. Biotinylated SEQ ID NO.1 (Biotin-LPc(XALNDE)R: monoisotopic mass (m/z) calculated: 1357.5; found: 1358.5, HPLC purity 94.5%.

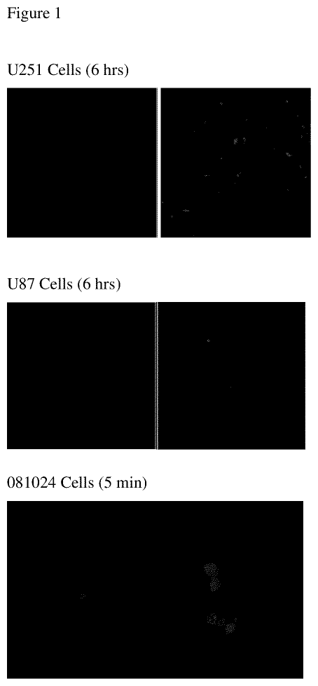

The ability of the four selected peptides to bind and/or enter GBM cells was evaluated. In a first set of experiments, adherent GBM cells and non-adherent GBM cells were exposed to 0.5 mM labelled peptides for 6 h, processed and analysed by confocal immunofluorescence microscopy. This experiment showed bright intracellular fluorescence for Biotinylated SEQ ID NO.1 as shown in FIG. 1. The control images (where cells are treated exactly the same, except they are not in contact with any peptide) are shown on the left hand image of each pair of images and ensured the specificity of the observed staining (nuclei can be seen but no Cy3 staining). Strikingly, the intracellular localization of the Cy3 staining (red) shown in the right hand image of each pair of images indicated that the cells had internalised the peptides.

In an attempt to capture membrane binding (only) the experiment was repeated with a shorter exposure of the cells to each peptide i.e. 5 minutes, the shortest practical timeframe. Interestingly, similar results were obtained, which confirmed that even at this earliest time point U251, U87 and 081024 cells had already internalised biotinylated SEQ ID NO.1.

Overall these results indicate that SEQ ID NO.1 is rapidly endocytosed (too fast for us to capture images where they are still localized at the plasma membrane/cell surface). The fact that peptides are seen inside the cells suggest an intracellular mechanism of action.

### Example 9: Effect of SEQ ID NO.1 on Cellular Invasion

To assess whether the ability of SEQ ID NO.1 to inhibit MMP-2 activity and, to a lesser extent, reduce the production of matrix proteinases and downregulate the expression of EMT markers, was of functional consequence in reducing the invasiveness of the GBM cells, the ability of the peptide was tested at the concentrations of 1, 10 and 100 μM (none of which are toxic to cells) to inhibit invasion of the GBM cells through a basement membrane. This assay, described in more detail below, is an in vitro surrogate for measurement of the in vivo invasion by tumour cells of their environment.

The cell invasion assay employs a simplified Boyden chamber-like design that consists of two chambers separated by a filter (an 8 μm polycarbonate (PC) membrane) coated with basement membrane or different extracellular matrix components. The cell suspension is placed in the top chamber and incubated in the presence of test media containing specific chemo-attractants in the bottom chamber. Cells migrate from the top chamber to the bottom of the filter by degrading the coating over the filter pores. Detection of cell invasion is quantified using Calcein AM. Cell dissociation/Calcein AM solution is placed in the bottom chamber to dissociate the migrating cells from the filter. Calcein AM is internalized by the cells, and intracellular-esterases cleave the aceto-methylester (AM) moiety. Free Calcein fluoresces brightly and is used to quantitate the number of cells that have invaded or migrated by comparison with a standard curve (In Vitro Technologies Pty Ltd, (https://lifescience.invitro.com.au/brands/r-and-d-systems/).

The filter was coated with PathClear® growth factor reduced BME. The selected peptides were applied to the cells in both upper and lower wells to make sure the concentration of peptides were the same and the chemoattractant was serum at a concentration of 2% (for adherent U87 & U251 cells) or growth factor mix required in the medium (for non- adherent, 081024 oncospheres).

Cell invasion was determined using CultureCoat® 96 Well Medium BME Cell Invasion assay. Briefly, the cells were pre-starved 24 h before harvest. Then seeding 25,000 per well of cell in the upper chamber with serum free medium together different concentration of peptide. The bottom chamber also added different concentration of peptides with 2% (V/V) serum for GBM adherent cells or growth factor mix for neurosphere cells as chemoattractant. After 24 h incubation, the invading cells were measured by Calcein AM and calculated based on standard curve.

The results are shown in Table 12, the results are expressed as % inhibition.

TABLE 12
Effect of peptide on cell invasion

| | Invasion Inhibition Index (%) | | | | | | | | |
|---|---|---|---|---|---|---|---|---|---|
| | U87 | | | U251 | | | 081024 | | |
| Pep- | 1 | 10 | 100 | 1 | 10 | 100 | 1 | 10 | 100 |
| tide | μM | μM | μM | μM | μM | μM | μM | μM | μM |
| Chlo- | 39.5 ± | — | — | 28.50 ± | — | — | 45.0 ± | — | — |
| ro- | 7.0 | | | 1.82 | | | 1.7 | | |
| toxin | | | | | | | | | |
| SEQ | 7.6 ± | 15.5 ± | 24.3 ± | 13.2 ± | 17.8 ± | 26.7 ± | 39.9 ± | 50.8 ± | 58.3 ± |
| ID | 4.7 | 4.4 | 5.3 | 5.0 | 6.3 | 4.7 | 1.6 | 2.3 | 2.0 |
| NO. | | | | | | | | | |
| 1 | | | | | | | | | |

The inhibition index of Chlorotoxin 1 μM is ˜30 to 45% depending on the cell line. The activity of SEQ ID NO.1 was fairly comparable to that of Chlorotoxin (at 1 μM) on the oncosphere (081024) cell lines. However, for U87 cells, SEQ ID NO.1 at 1 μM had lower inhibition index compared to Chlorotoxin.

### REFERENCES

Alves, T. R. et al., Glioblastoma cells: a heterogeneous and fatal tumor interacting with the parenchyma. Life Sci., 2011. 89(15-16): 532-539.
Conlon, G. A. and Murray, G. I. j. T. J o. p., Recent advances in understanding the roles of matrix metalloproteinases in tumour invasion and metastasis. Recent advances in Pathology; 2019. 247(5): 629-640.
Dai, C. and Holland, E. C.J. B. e. B. A. -R. o. C., Glioma models. Biochim. Biophys. Acta., 2001. 1551(1): M19-M27.
Goldbrunner, R., Bernstein, J. and Tonn, J. -C. J. A. n., Cell-extracellular matrix interaction in glioma invasion. Acta Neurochirurgica, 1999. 141(3): 295-305.
Iwadate, Y. J. O. L., Epithelial-mesenchymal transition in glioblastoma progression. 2016. Oncol. Lett., 11(3): 1615-1620
Kaufhold, S., Bonavida, B. J. J. o. E., and Research, C. C., Central role of Snail1 in the regulation of EMT and resistance in cancer: a target for therapeutic intervention. J. Exp. Clin. Cancer Res., 2014. 33(1): 62.
Kleihues, P., et al., Histopathology, classification and grading of gliomas. Glia, 1995. 15(3): 211-221.
McCawley, L. J. and Matrisian L. M. J. C. o. i. c. b., Matrix metalloproteinases: they're not just for matrix anymore! Curr. Opin. Cell Biol., 2001. 13(5): 534-540.
Nakagawa, T., et al., Secretion of matrix metalloproteinase-2 (72 kD gelatinase/type IV collagenase-=gelatinase A) by malignant human glioma cell lines: implications for the growth and cellular invasion of the extracellular matrix. J. Neurooncol. 1996. 28(1): 13-24.
Schuler, P., et al., Urokinase Plasminogen Activator, uPAR, MMP-2 and MMP-9 in the C6-Glioblatoma Rat Model. 2012, in vivo, 26: 571-576.
Van Meerloo, J., Kaspers, G. J. and Cloos, J., Cell sensitivity assays: the MTT assay, in Cancer cell culture. 2011. Cancer Cell Culture: Methods an dProtocols, Second Edition, Methods in Molecular Biology, Vol 731, Springer, p 237-245.

1. A compound of formula (I):
R₁-Xaa₁-Pro-c(Xaa₂-Xaa₃-Xaa₄-Xaa₅-Xaa₆-Xaa₇-Xaa₈-Xaa₉)-Xaa₁₀-R₂ (I)
wherein
R₁ is acyl or hydrogen;
R₂ is OH or NH₂;
Xaa₁ is a hydrophobic amino acid residue,
Xaa₂ is selected from SSA, Asp, Glu and Cys;
Xaa₃ is an amino acid residue having a small side chain;
Xaa₄ is a hydrophobic amino acid residue;
Xaa₅ is a polar uncharged amino acid residue;
Xaa₆ is a negatively charged amino acid residue;
Xaa₇ and Xaa₈ are each independently absent or an amino acid with a small side chain;
Xaa₉ is selected from SSA, Asp, Glu and Cys; and
Xaa₁₀ is a positively charged amino acid residue;
wherein one of the following applies:
i) Xaa₂ is SSA and Xaa₉ is Asp or Glu;
ii) Xaa₂ is Asp or Glu and Xaa₉ is SSA; or
iii) Xaa₂ and Xaa₉ are both Cys;
wherein Xaa₂ and Xaa₉ are linked to form a cyclic peptide; and wherein SSA is an amino acid residue of formula (II): 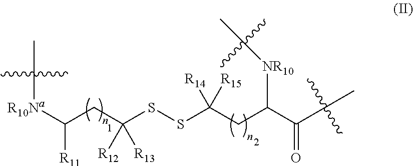
wherein
each R₁₀ is independently selected from hydrogen, substituted or unsubstituted C₁₋₆ alkyl;
R₁₁ is selected from hydrogen and methyl;
R₁₂, R₁₃, R₁₄ and R₁₅ are each independently selected from the group consisting of hydrogen and C₁₋₄ alkyl, or R₁₂ and R₁₃ taken together with the carbon atom to which they are attached and/or R₁₄ and R₁₅ taken together with the carbon atom to which they are attached for a cyclic structure selected from C₃₋₅ cycloalkyl and C₃₋₅ oxygen-containing, nitrogen containing or sulfur-containing heterocycle, each of which may be substituted or unsubstituted; and
n₁ and n₂ are each independently 0, 1, 2, 3 or 4;
with the proviso that at least one of R₁₂, R₁₃, R₁₄ and R₁₆ is not hydrogen;
wherein SSA is linked to Xaa₂ or Xaa₉ via N^{a};
or a pharmaceutically acceptable salt thereof.

2. The compound according to claim 1, wherein:
a. Xaa₂ is SSA and Xaa₉ is Asp or Glu,
b. Xaa₉ is Glu,
c. Xaa₂ is Asp or Glu and Xaa₉ is SSA,
d. Xaa₂ is Asp, or
e. Xaa₂ and Xaa₉ are both Cys and are linked to form a disulfide bond.

3.-6. (canceled)

7. The compound according to claim 1, wherein SSA is an amino acid residue of formula (III) or formula (IV): 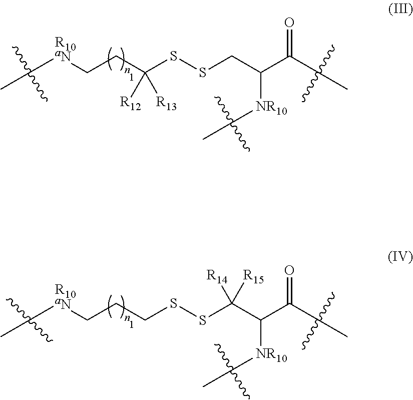
wherein R₁₀, R₁₁, R₁₂, R₁₃, R₁₄, R₁₅ and n₁ are as defined in formula (I).

8. The compound according to claim 1, wherein one or more of the following applies in relation to formula (II):
i) each R₁₀ is independently selected from hydrogen and methyl,
ii) R₁₁ is hydrogen;
iii) each R₁₂, R₁₃, R₁₄, R₁₅ are independently selected from hydrogen and C₁₋₃ alkyl;
iv) n₁ and n₂ are independently 0, 1 or 2.

9. The compound according to claim 1, wherein SSA is an amino acid residue of the formula (V):

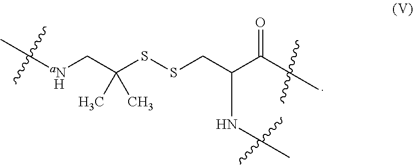

10. The compound according to claim 1, wherein one or more of the following applies in relation to formula (I):
i) R₁ is hydrogen;
ii) R₂ is NH₂;
iii) Xaa₁ is leucine, isoleucine, alanine or t-butylglycine;
iv) Xaa₃ is alanine;
v) Xaa₄ is leucine, isoleucine, alanine or t-butylglycine;
vi) Xaa₅ is asparagine or glutamine;
vii) Xaa₆ is aspartic acid or glutamic acid,
viii) Xaa₇ is absent or is glycine,
ix) Xaa₈ is absent or is glycine,
x) Xaa₁₀ is lysine or arginine.

11. The compound according to claim 10 wherein:
a. Xaa₁ is leucine or t-butylglycine,
b. Xaa₄ is leucine or t-butylglycine,
c. Xaa₅ is asparagine,
d. Xaa₆ is aspartic acid, and/or
e. Xaa₁₀ is arginine.

12.-15. (canceled)

16. The compound of formula (I) according to claim 1 selected from:
SEQ ID NO: 1
Leu-Pro-c(SSA*-Ala-Leu-Asn-Asp-Glu)-Arg-NH₂
SEQ ID NO: 2
Leu-Pro-c(SSA*-Ala-Leu-Asn-Asp-Glu)-Lys-NH₂
SEQ ID NO: 3
Leu-Pro-c(SSA*-Ala-Leu-Asn-Asp-Asp)-Lys-NH₂
SEQ ID NO: 4
Leu-Pro-c(SSA*-Ala-tBG-Asn-Asp-Glu)-Lys-NH₂
SEQ ID NO: 5
Leu-Pro-c(SSA*-Ala-tBG-Asn-Asp-Glu)-Arg-NH₂
SEQ ID NO: 6
tBG-Pro-c(SSA*-Ala-tBG-Asn-Asp-Glu)-Lys-NH₂
SEQ ID NO: 7
tBG-Pro-c(SSA*-Ala-tBG-Asn-Asp-Glu)-Arg-NH₂
SEQ ID NO: 8
tBG-Pro-c(SSA*-Ala-Leu-Asn-Asp-Glu)-Lys-NH₂
SEQ ID NO: 9
tBG-Pro-c(SSA*-Ala-Leu-Asn-Asp-Glu)-Arg-NH₂
SEQ ID NO: 10
Leu-Pro-c(SSA*-Ala-Leu-Asn-Asp-Gly-Gly-Glu)-Lys-
NH₂
SEQ ID NO: 11
Leu-Pro-c(SSA*-Ala-Leu-Asn-Asp-Gly-Gly-Glu)-Arg-
NH₂
SEQ ID NO: 12
Leu-Pro-c(SSA*-Ala-tBG-Asn-Asp-Gly-Gly-Glu)-Lys-
NH₂
SEQ ID NO: 13
Leu-Pro-c(SSA*-Ala-tBG-Asn-Asp-Gly-Gly-Glu)-Arg-
NH₂
SEQ ID NO: 14
tBG-Pro-c(SSA*-Ala-tBG-Asn-Asp-Gly-Gly-Glu)-Lys-
NH₂
SEQ ID NO: 15
tBG-Pro-c(SSA*-Ala-tBG-Asn-Asp-Gly-Gly-Glu)-Arg-
NH₂
SEQ ID NO: 16
tBG-Pro-c(SSA*-Ala-Leu-Asn-Asp-Gly-Gly-Glu)-Lys-
NH₂
SEQ ID NO: 17
tBG-Pro-c(SSA*-Ala-Leu-Asn-Asp-Gly-Gly-Glu)-Arg-
NH₂
SEQ ID NO: 18
Leu-Pro-c(SSA*-Ala-Leu-Asn-Asp-Gly-Gly-Asp)-Lys-
NH₂
SEQ ID NO: 19
Leu-Pro-c(SSA*-Ala-Leu-Gln-Asp-Gly-Gly-Asp)-Lys-
NH₂
SEQ ID NO: 20
Leu-Pro-c(SSA*-Gly-Leu-Asn-Asp-Gly-Gly-Asp)-Lys-
NH₂
SEQ ID NO: 21
Leu-Pro-c(SSA*-Ala-tBG-Asn-Asp-Gly-Gly-Asp)-Lys-
NH₂
SEQ ID NO: 22
Leu-Pro-c(SSA*-Ala-Leu-Gln-Asp-Gly-Gly-Asp)-Lys-
NH₂
SEQ ID NO: 23
Leu-Pro-c(Asp-Ala-Leu-Asn-Asp-SSA*)-Lys-NH₂
SEQ ID NO: 24
Ile-Pro-c(Asp-Ala-Leu-Asn-Asp-SSA*)-Arg-NH₂
SEQ ID NO: 25
Leu-Pro-c(Asp-Ala-Leu-Asn-Asp-SSA*)-Arg-NH₂
SEQ ID NO: 26
Ile-Pro-c(Asp-Ala-Ile-Asn-Asp-SSA*)-Arg-NH₂
SEQ ID NO: 27
Leu-Pro-c(Asp-Ala-Ile-Asn-Asp-SSA*)-Arg-NH₂
SEQ ID NO: 28
Leu-Pro-c(Asp-Ala-Leu-Asn-Glu-SSA*)-Arg-NH₂
SEQ ID NO: 29
Leu-Pro-c(Cys-Ala-Leu-Asn-Asp-Gly-Gly-Cys)-Lys-
NH₂
SEQ ID NO: 30
Leu-Pro-c(Cys-Ala-Leu-Asn-Asp-Gly-Gly-Cys)-Lys-
COOH.
wherein SSA* is a moiety of formula V as defined in claim 9.

17. A method of treating or preventing a cancer comprising administering a compound of formula (I) or a pharmaceutically acceptable salt thereof according to claim 1.

18. The method according to claim 17 wherein the cancer is a central nervous system cancer.

19. The method according to claim 18 wherein the central nervous system cancer is a brain cancer, a glioblastoma or a medulloblastoma.

20. (canceled)

21. The method according to claim 19 wherein the glioblastoma is glioblastoma multiforme.

22. A method of inhibiting urokinase plasminogen activator and/or a matrix metalloproteinase comprising contacting the urokinase plasminogen activator and/or matrix metalloprotease with a compound of formula (I) according to claim 1 or a pharmaceutically acceptable salt thereof.

23. The method according to claim 22 wherein the matrix metalloproteinase is MMP-2, MMP-9 or a mixture thereof.

24. A method of inhibiting tumor cell invasion in a brain tumor comprising administering to the brain tumor with a compound of formula (I) according to any one of claims 1 to 16 or a pharmaceutically acceptable salt thereof.

25. (canceled)

26. (canceled)

27. A compound of formula (I) according to claim 1 or a pharmaceutically acceptable salt thereof for use in treating cancer.

28. The compound for use according to claim 27 wherein the cancer is a central nervous system cancer.

29. (canceled)

30. A compound of formula (I) according to claim 1 or a pharmaceutically acceptable salt thereof for use in inhibiting tumor cell invasion in a brain tumor.


SEQUENCE LISTING (30 sequences)

SEQ ID NO: 1 - protein, 9 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa8 to form a cyclic peptide
  MISC_FEATURE (8)..(8): Xaa = Glutamic acid bonds to Xaa3 to form a cyclic peptide
  MISC_FEATURE (9)..(9): Xaa = Arg-NH2
LPXALNDXX

SEQ ID NO: 2 - protein, 9 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa8 to form a cyclic peptide
  MISC_FEATURE (8)..(8): Xaa = Glutamic acid bonds to Xaa3 to form a cyclic peptide
  MISC_FEATURE (9)..(9): Xaa = Lys-NH2
LPXALNDXX

SEQ ID NO: 3 - protein, 9 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa8 to form a cyclic peptide
  MISC_FEATURE (8)..(8): Xaa = Aspartic acid bonds to Xaa3 to form a cyclic peptide
  MISC_FEATURE (9)..(9): Xaa = Lys-NH2
LPXALNDXX

SEQ ID NO: 4 - protein, 9 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa8 to form a cyclic peptide
  MISC_FEATURE (5)..(5): Xaa = tertiary butyl glycine
  MISC_FEATURE (8)..(8): Xaa = Glutamic acid bonds to Xaa3 to form a cyclic peptide
  MISC_FEATURE (9)..(9): Xaa = Lys-NH2
LPXAXNDXX

SEQ ID NO: 5 - protein, 9 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa8 to form a cyclic peptide
  MISC_FEATURE (5)..(5): Xaa = tertiary butyl glycine
  MISC_FEATURE (8)..(8): Xaa = Glutamic acid bonds to Xaa3 to form a cyclic peptide
  MISC_FEATURE (9)..(9): Xaa = Arg-NH2
LPXAXNDXX

SEQ ID NO: 6 - protein, 9 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa8 to form a cyclic peptide
  MISC_FEATURE (5)..(5): Xaa = tertiary butyl glycine
  MISC_FEATURE (8)..(8): Xaa = Glutamic acid bonds to Xaa3 to form a cyclic peptide
  MISC_FEATURE (9)..(9): Xaa = Lys-NH2
XPXAXNDXX

SEQ ID NO: 7 - protein, 9 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa8 to form a cyclic peptide
  MISC_FEATURE (5)..(5): Xaa = tertiary butyl glycine
  MISC_FEATURE (8)..(8): Xaa = Glutamic acid bonds to Xaa3 to form a cyclic peptide
  MISC_FEATURE (9)..(9): Xaa = Arg-NH2
XPXAXNDXX

SEQ ID NO: 8 - protein, 9 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa8 to form a cyclic peptide
  MISC_FEATURE (8)..(8): Xaa = Glutamic acid bonds to Xaa3 to form a cyclic peptide
  MISC_FEATURE (9)..(9): Xaa = Lys-NH2
XPXALNDXX

SEQ ID NO: 9 - protein, 9 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa8 to form a cyclic peptide
  MISC_FEATURE (8)..(8): Xaa = Glutamic acid bonds to Xaa3 to form a cyclic peptide
  MISC_FEATURE (9)..(9): Xaa = Arg-NH2
XPXALNDXX

SEQ ID NO: 10 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Glutamic acid bonds to Xaa3 to form a cyclic peptide
  MISC_FEATURE (11)..(11): Xaa = Lys-NH2
LPXALNDGGXX

SEQ ID NO: 11 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Glutamic acid bonds to Xaa3 to form a cyclic peptide
  MISC_FEATURE (11)..(11): Xaa = Arg-NH2
LPXALNDGGXX

SEQ ID NO: 12 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (5)..(5): Xaa = tertiary butyl glycine
  MISC_FEATURE (10)..(10): Xaa = Glutamic acid bonds to Xaa3 to form a cyclic peptide
  MISC_FEATURE (11)..(11): Xaa = Lys-NH2
LPXAXNDGGXX

SEQ ID NO: 13 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (5)..(5): Xaa = tertiary butyl glycine
  MISC_FEATURE (10)..(10): Xaa = Glutamic acid bonds to Xaa3 to form a cyclic peptide
  MISC_FEATURE (11)..(11): Xaa = Arg-NH2
LPXAXNDGGXX

SEQ ID NO: 14 - protein, 11 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa10 to form a cyclic peptide
  MISC_FEATURE (5)..(5): Xaa = tertiary butyl glycine
  MISC_FEATURE (10)..(10): Xaa = Glutamic acid bonds to Xaa3 to form a cyclic peptide
  MISC_FEATURE (11)..(11): Xaa = Lys-NH2
XPXAXNDGGXX

SEQ ID NO: 15 - protein, 11 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa10 to form a cyclic peptide
  MISC_FEATURE (5)..(5): Xaa = tertiary butyl glycine
  MISC_FEATURE (10)..(10): Xaa = Glutamic acid bonds to Xaa3 to form a cyclic peptide
  MISC_FEATURE (11)..(11): Xaa = Arg-NH2
XPXAXNDGGXX

SEQ ID NO: 16 - protein, 11 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Glutamic acid bonds to Xaa3 to form a cyclic peptide
  MISC_FEATURE (11)..(11): Xaa = Lys-NH2
XPXALNDGGXX

SEQ ID NO: 17 - protein, 11 aa, Artificial Sequence
  synthetically derived, but based on cyclic peptides derived from Cuban Blue Scorpion
  MISC_FEATURE (1)..(1): Xaa = tertiary butyl glycine
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Glutamic acid bonds to Xaa3 to form a cyclic peptide
  MISC_FEATURE (11)..(11): Xaa = Arg-NH2
XPXALNDGGXX

SEQ ID NO: 18 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Aspartic acid bonds to Xaa3 to form a cyclic peptide
  MISC_FEATURE (11)..(11): Xaa = Lys-NH2
LPXALNDGGXX

SEQ ID NO: 19 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Aspartic acid bonds to Xaa3 to form a cyclic peptide
  MISC_FEATURE (11)..(11): Xaa = Lys-NH2
LPXALQDGGXX

SEQ ID NO: 20 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Aspartic acid bonds to Xaa3 to form a cyclic peptide
  MISC_FEATURE (11)..(11): Xaa = Lys-NH2
LPXGLNDGGXX

SEQ ID NO: 21 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (5)..(5): Xaa = tertiary butyl glycine
  MISC_FEATURE (10)..(10): Xaa = Aspartic acid bonds to Xaa3 to form a cyclic peptide
  MISC_FEATURE (11)..(11): Xaa = Lys-NH2
LPXAXNDGGXX

SEQ ID NO: 22 - protein, 11 aa, Artificial Sequence
  synthetically derived, but based on cyclic peptides derived from Cuban Blue Scorpion
  MISC_FEATURE (3)..(3): Xaa = S-((1-amino-2-methylpropan-2-yl)thio)-L-cysteine bonds to Xaa10 to form a cyclic peptide
  MISC_FEATURE (10)..(10): Xaa = Aspartic acid bonds to Xaa3 to form a cyclic peptide
  MISC_FEATURE (11)..(11): Xaa = Lys-NH2
LPXALQDGGXX

SEQ ID NO: 23 - protein, 9 aa, Artificial Sequence
  synthetically derived, but based on cyclic peptides derived from Cuban Blue Scorpion
  MISC_FEATURE (3)..(3): Xaa = Aspartic acid bonds to Xaa8 to form a cyclic peptide
  MISC_FEATURE (8)..(8): Xaa = S-((1-amino-2-methylpropan-2-yl)thio)-L-cysteine bonds to Xaa3 to form a cyclic peptide
  MISC_FEATURE (9)..(9): Xaa = Lys-NH2
LPXALNDXX

SEQ ID NO: 24 - protein, 9 aa, Artificial Sequence
  synthetically derived, but based on cyclic peptides derived from Cuban Blue Scorpion
  MISC_FEATURE (3)..(3): Xaa = Aspartic acid bonds to Xaa8 to form a cyclic peptide
  MISC_FEATURE (8)..(8): Xaa = S-((1-amino-2-methylpropan-2-yl)thio)-L-cysteine bonds to Xaa3 to form a cyclic peptide
  MISC_FEATURE (9)..(9): Xaa = Arg-NH2
IPXALNDXX

SEQ ID NO: 25 - protein, 9 aa, Artificial Sequence
  synthetically derived, but based on cyclic peptides derived from Cuban Blue Scorpion
  MISC_FEATURE (3)..(3): Xaa = Aspartic acid bonds to Xaa8 to form a cyclic peptide
  MISC_FEATURE (8)..(8): Xaa = S-((1-amino-2-methylpropan-2-yl)thio)-L-cysteine bonds to Xaa3 to form a cyclic peptide
  MISC_FEATURE (9)..(9): Xaa = Arg-NH2
LPXALNDXX

SEQ ID NO: 26 - protein, 9 aa, Artificial Sequence
  synthetically derived, but based on cyclic peptides derived from Cuban Blue Scorpion
  MISC_FEATURE (3)..(3): Xaa = Aspartic acid bonds to Xaa8 to form a cyclic peptide
  MISC_FEATURE (8)..(8): Xaa = S-((1-amino-2-methylpropan-2-yl)thio)-L-cysteine bonds to Xaa3 to form a cyclic peptide
  MISC_FEATURE (9)..(9): Xaa = Arg-NH2
IPXAINDXX

SEQ ID NO: 27 - protein, 9 aa, Artificial Sequence
  synthetically derived, but based on cyclic peptides derived from Cuban Blue Scorpion
  MISC_FEATURE (3)..(3): Xaa = Aspartic acid bonds to Xaa8 to form a cyclic peptide
  MISC_FEATURE (8)..(8): Xaa = S-((1-amino-2-methylpropan-2-yl)thio)-L-cysteine bonds to Xaa3 to form a cyclic peptide
  MISC_FEATURE (9)..(9): Xaa = Arg-NH2
LPXAINDXX

SEQ ID NO: 28 - protein, 9 aa, Artificial Sequence
  synthetically derived, but based on cyclic peptides derived from Cuban Blue Scorpion
  MISC_FEATURE (3)..(3): Xaa = Aspartic acid bonds to Xaa8 to form a cyclic peptide
  MISC_FEATURE (8)..(8): Xaa = S-((1-amino-2-methylpropan-2-yl)thio)-L-cysteine bonds to Xaa3 to form a cyclic peptide
  MISC_FEATURE (9)..(9): Xaa = Arg-NH2
LPXALNEXX

SEQ ID NO: 29 - protein, 11 aa, Artificial Sequence
  synthetically derived, but based on cyclic peptides derived from Cuban Blue Scorpion
  MISC_FEATURE (3)..(3): Xaa = Cysteine cyclized by disulfide bond with Xaa10
  MISC_FEATURE (10)..(10): Xaa = Cysteine cyclized by disulfide bond with Xaa3
  MISC_FEATURE (11)..(11): Xaa = Lys-NH2
LPXALNDGGXX

SEQ ID NO: 30 - protein, 11 aa, Artificial Sequence
  synthetically derived, but based on cyclic peptides derived from Cuban Blue Scorpion
  MISC_FEATURE (3)..(3): Xaa = Cysteine cyclized by disulfide bond with Xaa10
  MISC_FEATURE (10)..(10): Xaa = Cysteine cyclized by disulfide bond with Xaa3
LPXALNDGGXK